(19) United States
(12) Patent Application Publication
(10) Pub. No.: US 2003/0134412 A1
(43) Pub. Date: Jul. 17, 2003

(54) Method for isolating plasmids from suspended bacterial or yeast cells

(76) Inventors: Thomas Kolzau, Hamburg (DE); Wilhelm Pluster, Boulder, CO (US)

Correspondence Address: RANKIN, HILL, PORTER & CLARK, LLP, 700 HUNTINGTON BUILDING, 925 EUCLID AVENUE, SUITE 700, CLEVELAND, OH, 44115-1405, US

(21) Appl. No.: 10/297,367

(22) PCT Filed: Mar. 18, 2002
(86) PCT No.: PCT/EP02/02964

(30) Foreign Application Priority Data
Mar. 21, 2001 (DE) 101 13 815.6

(51) Int. Cl. C12N 15/74; C12N 1/08

(52) U.S. Cl. 435/320.1; 435/270

(57) ABSTRACT

The invention relates to a method for isolating plasmids from suspended bacterial or yeast cells using a filter matrix, at least one lysing substance being added to the suspension and pre-damaging or completely lysing the predominant portion of the suspended cells, at least one conformation-altering substance being added to said suspension to alter the conformation of the plasmids to be isolated in such manner as to promote retention of the cell components in or at the filter matrix, the suspension thereupon being moved through a filter matrix and the cells if not yet lysed then being lysed to completion upon contact with the filter matrix and the released plasmids being retained in or at the said filter matrix.

The invention relates to a method for lysing bacterial or yeast cells and then isolating the lysis-released plasmids using a filter matrix.

Such procedures are carried out routinely on different kinds of cells.

A common application is the isolation of plasmids from bacterial cells. In one conventional procedure, the bacteria are pelletized and the pellet is then re-suspended by adding a first buffer. Thereupon another buffer is added to the suspension to implement cell lysis. Almost always this buffer will be strongly alkaline and contain highly effective detergents, for instance sodium dodecyl sulfate (SDS). Lastly still another buffer is added to neutralize the suspension again. This three-step buffer addition also is called alkaline lysis.

In a further step, the suspension illustratively will be filtered. A clear filtrate containing the plasmids is obtained. A binding buffer containing chaotropic reagents and silica particles is added to the filtrate. The chaotropic reagents contained in the binding buffer assure that the plasmids shall selectively bond to the silica particles.

Following silica particle washing, the plasmids may be eluted from the silica particles by an elution buffer and thereupon will be available in pure form.

The described cell processing until binding the plasmids to the silica matrix requires using several different buffers and also carrying out a substantial series of different steps of centrifuging, filtering, decanting and pipetting. Such a procedure is time consuming and expensive and moreover hampers automation.

The objective of the invention is to create a simplified method for isolating plasmids.

The objectives of the invention are attained using methods exhibiting the features of the invention of claims 1 and 2.

The cells are first lysed, as in the state of the art. The buffer of the invention used for that purpose contains at least one lysing substance attacking the cells being processed and at least destabilizing them. Preferably the buffer's composition is such as to damage the cells without the cell contents being discharged. However the invention also shall cover the feasibility of complete cell lysis when mixing the cells with the buffer. In both modes the cells of course also may first be suspended in a buffer devoid of lysing substances, said substances in that event being separately added in a subsequent step.

It is understood that the above discussion must be considered statistically. In both modes the less desired of the two effects will also be present up to a given percentage, that is, the first mode cannot preclude the presence of fully lysed cells besides the pre-damaged ones, and vice versa. Accordingly when hereafter discussing lysed or damaged cells, what shall be meant is that at least the predominant portion of the cells will be in that state.

Preferably the buffer of the invention also contains one or more substances that shall alter the plasmids' conformations in such a way that these plasmids shall be retained better, or at all, in the filter matrix.

A preferred buffer of the invention used for isolating plasmids from bacterial cells illustratively may contain a hyper- or a hypo-osmotic salt concentration and furthermore may contain lysozyme and precipitation agents, preferably polyethyleneglycol (PEG), but also polyvinylpyyrolidone (PVP), ethanol or isopropanl or chaotropic salts such as guanidine isothiocyanate or urea.

When used in conjunction with the adjusted salt concentration, the use of lysozyme entails damaging the bacterial cells or their lysis. PEG on the other hand entails that the plasmids which following cell lysis shall exit such cells will undergo condensation and then can be retained in condensed form in the pores of a conventional filter matrix.

The term “condensation” herein denotes alterations in conformation due to external factors, including plasmid compression as far as the insoluble state. Plasmid compression is concomitant with decreasing flexibility and increasing complexity.

Obviously the invention is also meant to include the feasibility of adding the minimum of one conformation-altering substance separately, that is not jointly with the minimum of at least one lysing substance. The sole significant constraint in this regard is that the conformation-altering substance shall be added before the suspension, or a cell fraction containing the pertinent plasmids, shall be moved through the filter matrix.

Within the scope of this application, the expression “suspension” applies essentially when cells which are intact or merely are pre-damaged are present. If on the other hand the cells are fully lysed, the proper terminology will be “cell fractions”.

In the event that the cells initially only are pre-damaged, the invention provides furthermore that the suspension illustratively is centrifuged onto a filter matrix or aspirated under vacuum through a filter matrix. In this process the cells will be forced in widely intact form against the filter matrix. Because of their prior damage, the cells are very sensitive to mechanical stresses. The shearing forces arising at contact with the filter matrix almost always suffice therefore to tear open the pre-damaged cells.

Thereupon, in the zone of the filter matrix, the cells release their contents, and, as indicated above, for instance the plasmids to be isolated undergo the desired conformation alteration on account of the PEG for instance contained in the buffer.

In the event the cells will be completely lysing already after contact with the initially added minimum of one lysing substance, processing may be changed somewhat according to one preferred implementation. In this case lysing may be followed by a first step, for instance centrifuging, wherein the cell debris will be removed. The cell fraction so obtained can—if not already done—be mixed with conformation-altering substance of the invention and then be moved through the filter matrix.

In both above described approaches, the filter matrix may thereupon be washed in order to remove other interfering cell components. Then elution shall take place preferably using a buffer reversing the conformation alteration of the plasmids to be isolated, which then shall be again released from the filter matrix.

The filter matrix' structure, or its pore diameter, are so matched in the invention to the cell components to be isolated, that is they are selected in such manner, that the plasmids may be retained in the matrix in the conformation constrained by the PEG, on the basis of which they may be retained selectively in the filter matrix.

One substantial advantage of the invention relative to the state of the art's known methods is that the lysis of the cells may be carried out under non-denaturing or only weakly denaturing conditions. As a result the released cell ingredients remain in solution following lysis. The solution containing the cell ingredients may be fed in the absence of intermediate steps of purification and renaturing to a filter to carry out selective isolation for instance of the plasmids. Under the conditions adjusted in the manner of the invention, the plasmids shall be retained at or in the filters while the other ingredients pass through it.

In a further variation of the invention, already after contact with the minimum of at least one lysis substance, the cells already shall be lysed completely, the conditions in this case being that they shall not be or only weakly denaturing.

As regards this variation of the invention, and following lysis, a step may be inserted, for instance a centrifuging step, during which the cell debris shall be removed. The cell fraction produced in this manner may be mixed, unless they already were, with the minimum of one conformation-altering substance of the invention which selectively lowers the plasmids' solubility, and then are moved through the filter matrix.

As regards the initially cited, so-called “alkaline lysis” used in routine procedures, said lysis entails denaturing the released ingredients and the plasmids must be renatured in further processing, in turn requiring depending on the protocols also a centrifuging step and/or a filtering step in order that the renatured plasmids be separated from the denatured proteins and further ingredients.

The patent document EP 0,473,575 relates to improving the known “alkaline lysis” wherein, using a special buffer, lysis, neutralization and deproteination take place simultaneously. But this document also requires subsequent purification steps before the actual purification (isolation) of the DNA may take place.

Compared with these known procedures, that of the invention may be carried out in significantly simplified manner. The steps of renaturing and ensuing centrifuging may be eliminated entirely. The invention actually allows carrying out lysing and subsequent plasmid isolation without intermediate centrifuging or other separation steps.

As regards the two above described approaches, conventionally the filter matrix shall then be washed to remove other, interfering cell components. Subsequently elution with a buffer is carried out then to reverse the change of conformation of the plasmids to be isolated which then are again released by the filter matrix.

The design of the filter matrix, i.e. the diameter of its pores, are matched in the invention to the cell components to be isolated, that is they are selected in such a way that the plasmids may be retained in the matrix in the confirmation constrained by the PEG.

The method of the invention offers a number of advantages. One essential advantage is that in the simplest case of (re)-suspension of the cells to be processed until the cell components have been isolated at the filter matrix, only one buffer need be used. In this case the operational steps merely are the preparation of the suspension and centrifuging or filtering.

Compared to the procedures of the known state of the art, the method of the invention allows saving time and hence costs. Method automation is feasible in simple manner.

Appropriate buffer compositions as well as suitable filter matrices are discussed in the Examples farther below.

It must be stressed again in a general way that any buffer shall be suitable which contains substances able to damage the cells, including those lysing them and/or altering the conformation of the plasmids to be isolated.

The filter matrix is selected in such a way that when there is contact between the pre-damaged cells and the matrix, the entailed mechanical forces are able to fully lyse the cells and in that, following conformation alteration, the cell components to be isolated can be retained in or at the matrix.

Surprisingly it was found that a large number of different filter materials may be used. It appears that the composition or the surface nature of the filter matrix will not significantly affect the processing by the method of the invention. In another words, in most cases pre-damaged cells will tear open independently of the filters used when coming into contact with the filter surfaces.

Even though the method of the invention operates fairly independently of the particular filter matrices being used, the results however may be optimized by appropriately structuring said matrices.

In this respect an advantageous design consists of a two-layer filter matrix. One layer—the upper one—constitutes the surface with which the pre-damaged cells shall be in contact and said contact then entails full lysing. Also the comparatively large cell debris will be retained in the upper layer following lysing.

On the other hand the significant plasmids shall be collected in the filter matrix' lower layer.

In an especially preferred implementation of the invention, the surface structures—hereafter topographies—of the lower filter layer may be adjusted by means of the ambient buffer medium. Illustratively the topographies may be affected by the same buffer substance(s) implementing the desired conformation alterations for the plasmids to be isolated. The topographies illustratively may exhibit tentacles that shall change the cross-sections of the filter matrix' pores as a function of the buffer being used. If the tentacles are straightened out, the pores shall be transmitting. If on the other hand they are compressed, they shall constrict the pore cross-sections and impede transmission. A membrane with a functionalized surface such as known for instance from the German patent documents 10106199.4 or 10006590.2 may be used in this regard.

If the elution buffering conditions are changed, the topographies of the lower filter layer and the cell components may be controlled thereby to allow easier detachment.

The upper filter layer also may be in the form of a surface-subtending, i.e. a “topological” membrane. It is moreover conceivable to constitute said upper filter layer only during the centrifuging or filtering step. For that purpose the buffer shall comprise beads which for instance during centrifuging shall sediment first and then shall constitute the upper filter layer, however they may also form, in case only one layer shall be used, the entire filter matrix.

The following Examples elucidate the invention's embodiment variants under different conditions.

A. EMPLOYED BUFFERS

Buffer PI (hyper-osmotic, prelytic)

50 mM Tris ph 8.0

10 mM EDTA ph 8.0

(1.5% Triton-X-100)

(10-20% saccharose)

50 μg/ml RNase A

0.4 g/ltr lysozyme

pH 8.0

Buffer II (hypo-osmotic, prelytic)

ddH₂O

(1% Triton X-100)

0.3 g/ltr lysozyme

1 mM Tris

50 μg/ml RNase A

(0.4 g/ltr lysozyme)

Buffer PIII (lysing buffer)

50-100 mM Tris

10-50 mM EDTA

(10-20% saccharose)

1-5% Triton X-100

100-200 μg/ml RNase A

0.8-8 g/ltr lysozyme

(0.1-1% SDS)

(proteinase K)

pH 8.0-10.5

The buffers PI-PIII are always used in conjunction with 12-20% PEG (alternatively PVP, isopropanol etc)., 2.5 M NaCl in the ratio of 1:1. Moreover beads of different kinds may be added. In the latter case the designations PI-PIII shall be complemented by also denoting the corresponding beads.

B FILTER MATRICES

a) single-layer

PVDF 0.45 μm

PVDF 0.5 μm

glass fiber/C

glass fiber/F

nylon 0.45 μm

nylon 0.5 μm

PKB(B)

nylon 0.45 with chemically functionalized surface

b) two-layer

glass fiber/F+MBP support

glass fiber/C 2-ply

nylon 0.2-1.2 μm+cellulose

c) beads or “fluid filter”

Various beads were tested for their suitability as filter layers with respect to sedimentation.

B(1) Davisil 200-425, silica pore size 150 Å, particle size 33-75 μm

B(2) Sephadex G200, dextran crosslinked with epichlorohydrin, particle size 25-100 μm

B(3) Sephadex LH20, particle size 20-100

B(4) Celatom FW14, cellulose

B(5) Celatom FW50, cellulose

B(6) Celatom FW60, cellulose

B(7) Celatom FW80, cellulose

B(8) AFR25PDB-SO₃, CK10M/4PDB-SO0₃, polystyrene crosslinked with divinylbenzene, ion exchanger, particle size about 20 μm,

B(9) POROSR1-10, polystyrene crosslinked with divinylbenzene

B(10) POROSR1-20, polystyrene crosslinked with divinylbenzene

B(11) POROSR2-10, polystyrene crosslinked with divinylbenzene

B(12) POROSR2-20, polystyrene crosslinked with divinylbenzene controls:

B(13) glass beads

B(14) no beads

B(15) empty

B(16) Perfect Prep Mini.

### EXAMPLE 1

### Isolating Different Plasmids from Different E. Coli Strains

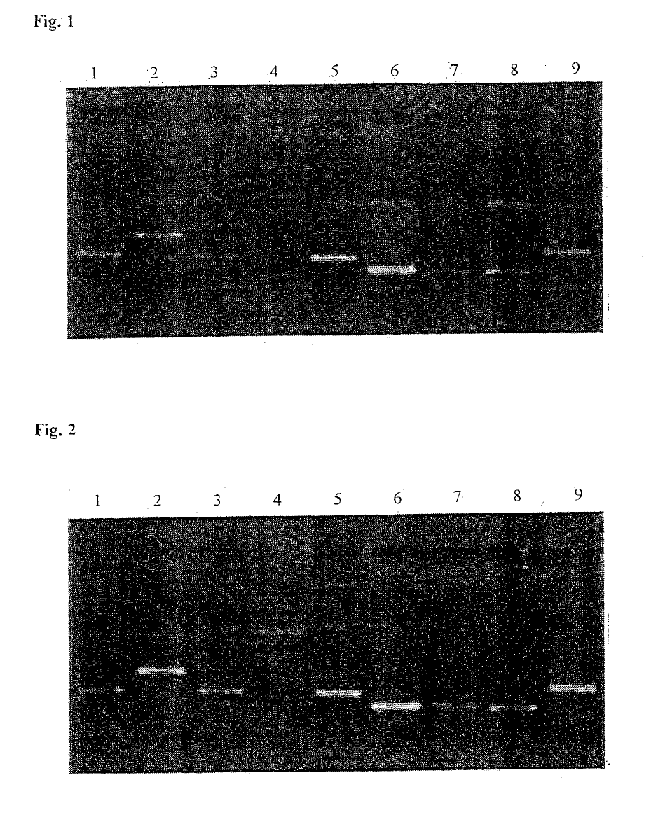

The cells always were cultured in 1.2 LB medium and then pelletized. The cell pellets then were placed in 400 μl of buffer I+30 μl glass beads and the suspension so made was forced by centrifuging through a single layer filter matrix (nylon 0.45) into single columns. After washing with ethanol, elution was carried out in each case using 30 μl TE buffer and the eluate was analyzed by gel-electrophoresis. The results are shown in FIG. 1 and were attained using conventional procedures (Mini-Prep Eppendorf).

It is seen that the method of the invention (FIG. 2) substantially produces the same bands as the conventional procedure (FIG. 1), however there are substantially fewer procedural steps in the method of the invention. The association between the lanes and the plasmids to be isolated is as follows: lane 1: pbluescript, (XL1blue); lane 2: pUC18 (XL1blue); lane 3: Q1.2 (XL1blue); lane 4: pBR322 (XL1blue); lane 5: GAPDH (HB101); lane 6: pbluescript (DH10β); lane 7: pbluescript (DH5α); lane 8: pUC19 (JM109) and lane 9: hsp70(XL1blue).

### EXAMPLE 2

### Isolating Plasmids Using Different Two-Layer Filter Matrices

1.2 ml of LB bacterial cultures were pelletized and then re-suspended each time with 400 μl buffer I. After being transferred into 96-well plates, the pre-lysate was moved by centrifuging through a two-layer matrix consisting of a cellulose based material and nylon of pore widths of 0.2-1.2 μm.

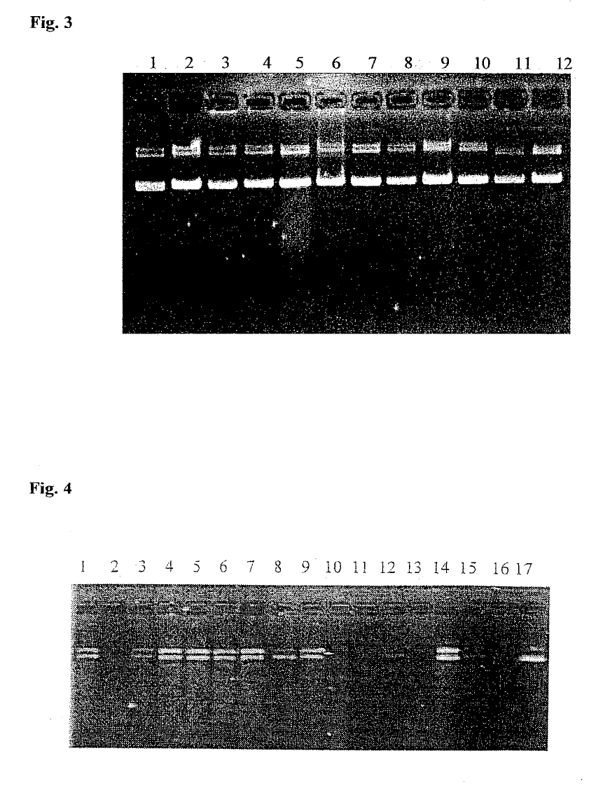

After washing in ethanol, elution is carried out using 30 μl TE buffer. FIG. 3 shows the gel-electrophoresis analysis of the preparations compared to a conventional plasmid mini preparation using the Perfect Prep Plasmid Minikit of Eppendorf Co. Comparison with the control reveals a qualitative and quantitative success in preparation in the manner of the method of the invention while reducing procedural complexity.

### EXAMPLE 3

### Isolating Plasmids Using Different Beads As Fluid Filters

1.2 ml of LB bacterial cultures were pelletized and then are mixed each time with 400 μl of buffer I and then were re-suspended each time with 30 μl of different types of beads. After being transferred into 96-well plates, the pre-lysate was moved by centrifuging through a single-layer glass matrix.

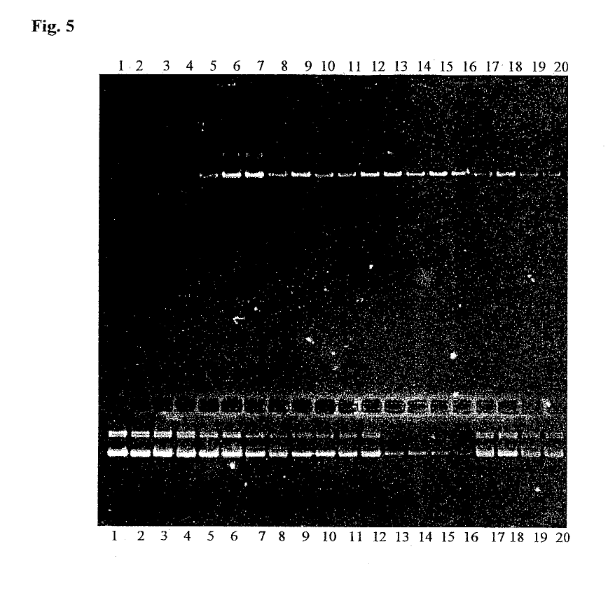

Following washing in ethanol, elution is carried out with 30 ml of TE buffer. FIG. 4 shows the gel-electrophoresis analysis of the preparations compared to a conventional plasmid mini preparation using the Perfect Prep Plasmid Minikit made by Eppendorf Co. This comparison shows that, in principle, different beads may be used, but that their differences will lead to different yields depending on their particular properties. The following beads were used: (1) Davisil 200-245, silica pore size 150 Å, particle size 33-75 (2) Sephadex G200, dextran crosslinked with epichlorohydrin, particle size 25-100 μm, (3) Sephadex LH20, particle size 20-100, (4) Celatom FW14, cellulose, (5) Celatom FW50, cellulose, (6) Celatom FW60, cellulose, (7) Celatom FW 80, cellulose, (8) AFR25PDB-SO₃, (9) CK10M/4PDB-SO0₃, polystyrene crosslinked with divinylbenzene, ion exchanger, particle size about 20 μm, (10) POROSR1-10, polystyrene crosslinked with divinylbenzene, (11) POROSR1-20, polystyrene crosslinked with divinylbenzene, (12) POROSR2-10, polystyrene crosslinked with divinylbenzene, (13) POROSR2-20, polystyrene crosslinked with divinylbenzene controls, (14) glass beads, (15) without glass beads, (16) empty, (17) Perfect Prep Mini.

### EXAMPLE 4

### Isolating Plasmids Using Different Filter Matrices

In each case the cells were cultivated in an LB medium and then pelletized. The cell pellets then were placed each time in 400 μl buffer I+30 μl glass beads and the suspension so prepared was pressed by means of a vacuum or centrifuging through single-layer and two-layer filter matrices into 96-well plates. After washing with ethanol, 30 μl of TE buffer were eluted each time and the eluates were analyzed by gel electrophoresis (FIG. 5).

Accordingly the method of the invention leads to successful preparation while using different matrices. The association between lanes and filter matrices (each time four parallel lines) is as follows:.

Lanes at the top:

Lanes 1-4=PVDF 0.45 μ; lanes 5-8=glass fiber/C; lanes 9-12=glass fiber/F;

lanes 13-16=surface-modified nylon; lanes 17-20=glass fiber/F+MBD;

Lanes at the bottom:

Lanes 1-4=PVDF 0.5μ; lanes 5-8=PKB(B); lanes 9-12=nylon 0.4 μ; lanes 13-16 =glass fiber X; lanes 17-20=glass fiber/c 2-ply.

### EXAMPLE 5

### Isolating Plasmids When The Cells Are Completely Lysed In A First Step

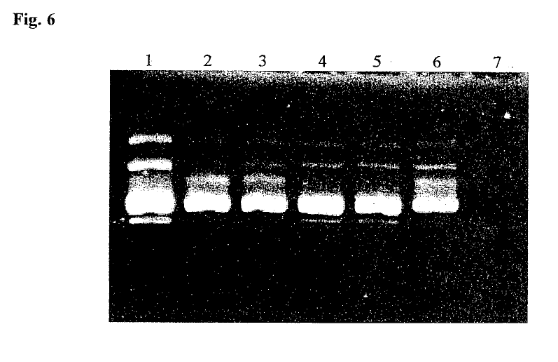

The cells were cultured each time in 1.2 ml of LB medium and then pelletized. The cell pellets then were placed in each case in 400 μl buffer I+30 μl beads (pre-lysis) or in buffer IV (lysis) and the suspension so prepared was forced by centrifuging through a single-layer filter matrix into individual columns (A), or the suspension was pelletized on the precursor path. The results are shown in FIG. 6. Control (lane 1) Perfect Prep Minikit; Complete lysis: (lanes 2+3) lysing conditions (buffer IV+30 μl beads)+filter matrix as in (A), lanes (4+5) lysing conditions (buffer IV+beads) without filter matrix, cell debris were centrifuged off, and then the excess is prepared again using a filter matrix as in (B). Pre-lysing: (lane 6) buffer I+ beads through the filter matrix as in (A), (lane 7) buffer I+ beads, after centrifuging only the supernatant was prepared as in (lanes 3+4) or (B) through a filter matrix. The results show that the use of different buffers does allow complete lysis or pre-lysis cell embrittling, such embrittling requiring mechanical shearing forces to attain complete lysis.

### EXAMPLE 6

### Subsequent Application/Sequencing Quality of DNA Plasmid

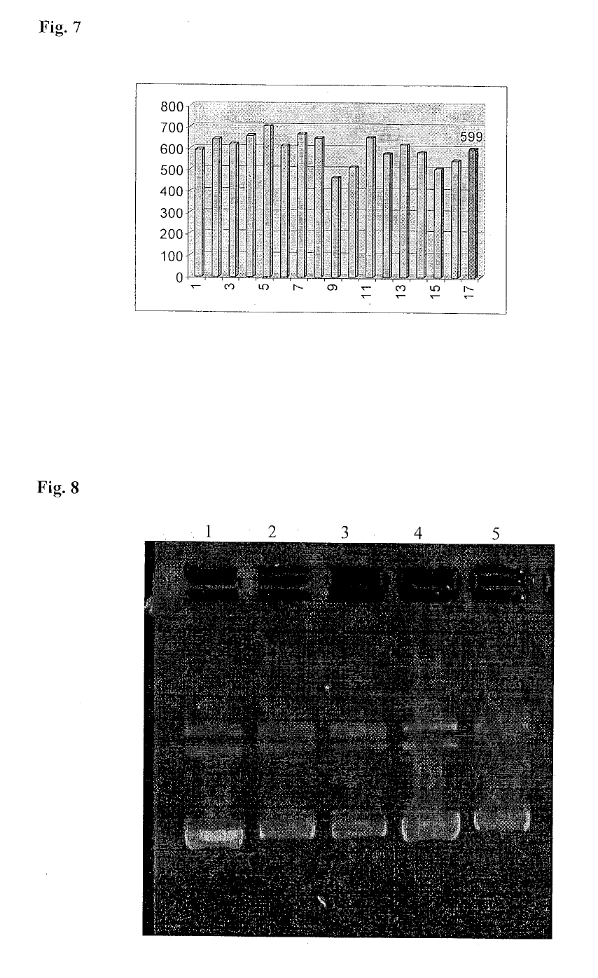

Each time, the bacteria were cultured in 1.2 LB medium and then pelletized. The cell pellets each time were placed in 400 μl of buffer I+30 μl glass beads and the suspension so prepared was centrifugally pressed through a single-layer glass filter matrix. Following washing in ethanol, the DNA plasmid was eluted using 30 μl of TE buffer and aliquots each of 4 μl were analyzed by automated capillary sequencing and the phred20 scores were ascertained (FIG. 8). As shown by the results, the DNA plasmid preparation of the present application allows good sequencing which is identical to a highly pure control (see bar 17).

### EXAMPLE 7

### Lysing Under Hypo-Osmotic Conditions

Each time, bacteria were cultured in 1.2 LB medium and then pelletized. The cell pellets each time were placed in 400 μl of buffer I and buffer II+30 μl glass beads and the suspension so prepared was forced by centrifuging through a single-layer glass filter matrix. After washing with ethanol the DNA plasmid was eluted with 30 μl of TE buffer and the eluate was analyzed by gel electrophoresis (FIG. 9; lane 1: MiniPrep; lane 2: buffer I; lane 3: buffer II; lane 4: MiniPrep; lane 5: buffer II). It was found that the conditions for pre-lysing with subsequent lysis at the matrix can be carried out both using a hypo-osmotic and a hyper-osmotic composition.

1. A method for isolating plasmids from suspended bacterial or yeast cells using a filter matrix, wherein
at least one lysing substance is added to the suspension, said substance comprising, in predominant proportion, pre-damaged though not fully lysed cells,
at least one conformation-altering substance is added to the suspension and implements a change in conformation of the plasmids to be isolated in a manner to promote retention of cell components in or at the filter matrix,
the suspension is thereupon moved through a filter matrix in contact with which the cells are completely lysed and the released plasmids are retained in or at this filter matrix.

2. A method for isolating plasmids from suspended bacterial or yeast cells using a filter matrix, wherein
at least one lysing substance is added to the suspension and implements complete cell lysing,
where called for, the cell debris are removed,
at least one conformation-altering substance is added to the suspension and implements a change in conformation in the plasmids to be isolated in such manner that retention of the cell components in or at the filter matrix is promoted,
the supernatant containing the cell components to be isolated then is moved through the filter matrix, the plasmids to be isolated being retained in or at the filter matrix.

3. Method as claimed in either of claims 1 and 2, characterized in that the conformation-altering and lysing substances are jointly added in a suspension buffer.

4. Method as claimed in one of claims 1 through 3, characterized in that the conformation-altering substance is PEG (polyethyleneglycol).

5. Method as claimed in one of claims 1 through 6, characterized in that enzymes and/or mild detergents and/or osmotic substances, in particular salts or sugars, are among the employed substances acting in lysing manner and causing pre-damage to the cells and applying either a hypo-osmotic or a hyper-osmotic stress to said cells.

6. Method as claimed in one of claims 1 through 5, characterized in that the method employs a filter matrix comprising two filter layers.

7. Method as claimed in one of claims 1 through 6, characterized in that the filter matrix or at least one of the filter layers comprises ambient-dependent surface structures.

8. Method as claimed in one of claims 1 through 7, characterized by adding beads which, in the course of processing, in particular as a consequence of a centrifuging or a filter step, will collect at the filter matrix or at the upper layer of a filter matrix.

9. Method as claimed in claim 8, characterized in that, jointly with the minimum of conformation-altering and/or the minimum of one lysing substance, the beads are contained in one common buffer.

11. Method as claimed in one of claims 1 through 10, characterized in that a buffer is used to elute the cell components from the filter matrix and reverses the conformation-alteration of the plasmids to be isolated.
